(19) United States
(12) Patent Application Publication
(10) Pub. No.: US 2023/0057086 A1
(43) Pub. Date: Feb. 23, 2023

(54) PROVISION OF BACTERIOPHAGES IN VARIOUS DOSAGE FORMS AND BACTERIOPHAGE APPLICATION DEVICE

(71) Applicant: PhaTEC GmbH, Kiel (DE)

(72) Inventors: Simon Frank Junghans, Kiel (DE); Justus Gross, Lutterbek (DE)

(21) Appl. No.: 17/792,885

(22) PCT Filed: Nov. 21, 2020
(86) PCT No.: PCT/DE2020/100988

§ 371 Date: Jul. 14, 2022

(30) Foreign Application Priority Data
Jan. 14, 2020 (DE) 10 2020 100 725.6

(51) Int. Cl. A61K 35/76; A61K 9/06; A61K 9/48; A61K 9/00; A61L 17/00; A61L 26/00; C12N 7/00

(52) U.S. Cl. CPC A61K 35/76; A61K 9/06; A61K 9/0078; A61K 9/4808; A61L 17/005; A61L 26/0066; C12N 7/00; A61L 2300/404; C12N 2795/00

(57) ABSTRACT

The invention relates to bacteriophage means, namely intracorporal bacteriophage means, nasopharyngeal and pulmonary bacteriophage means, cutaneous bacteriophage means, and bacteriophage suture means, and in addition a two-syringe bacteriophage means, a nasopharyngeal and pulmonary bacteriophage means device, and a bacteriophage sensitive testing application.

The invention relates to bacteriophage means, namely intracorporal bacteriophage means, nasopharyngeal and pulmonary bacteriophage means, cutaneous bacteriophage means and bacteriophage suture means, and moreover a two-syringe bacteriophage means, a nasopharyngeal and pulmonary bacteriophage means device and a bacteriophage sensitive testing application.

Bacteriophages, or briefly BPH or phages, are various groups of viruses that are specific to bacteria as host cells, i.e., they specifically affect bacteria. This means that the host, for example a mammal and especially a human, will not be affected. Infection of bacteria or other pathogenic microorganisms by virulent phages results in the lytic cycle and ultimately to lysis and destruction of the bacteria. Endotoxins are bacterial toxins that, contrary to exotoxins, are not secreted by living bacteria but are only released by autolysis.

Phages have found a wide range of applications in medicine, veterinary medicine, biology, agricultural sciences, food processing, and especially in the field of genetic engineering. For example, phages are used in medicine to identify bacterial pathogens due to their host specificity. The use of phages in therapy of bacterial infections was discovered by Felix d’Hérelle long before the discovery of penicillin and antibiotics. Later, however, with the introduction of chemotherapy by antibiotics, phage therapy was deemed impractical and fell into oblivion. Due to the increasing incidence of multiple antibiotic resistance, intensive research is currently being conducted again on the use of bacteriophages as antibiotic substitutes in human medicine.

Bacteriophages can be obtained from nature. For this purpose, water samples, blood samples, swabs, human or animal secretions, or other samples are taken and spread on nutrient plates. By incubating these plates (36° C. - 37° C. for 24 h), existing bacteriophages are found, as indicated by lysis areas. By detecting the prokaryotes which are present, an initial statement can be made as against which bacterium the phage found is lytically active.

For purification, the plaque in the bacterial lawn is excised and vortexed for at least 10 minutes in a snap cap tube containing liquid nutrient solution. The liquid nutrient solution is removed, sterile-filtered and applied to a nutrient medium plate previously inoculated with the appropriate germ and reincubated for another 24 h at 36° C. - 37° C. A plaque is again excised and processed as described. This cycle should be repeated at least 5 times to ensure that the isolated phages present are only clones of one phage.

To produce larger quantities of phage clones of one phage, the sterile-filtered phage solution from the snap cap tube is placed on an inoculated plate after purification at least 5 times and incubated again as described. Extraction buffer is then added to the plate, which is agitated on the culture medium through a shaking apparatus for 30 minutes. The extraction buffer is removed and sterile-filtered, yielding a sterile phage solution.

A bacteriophage solution can be stabilized by adding stabilizers such as: CaC1₂ and stabilized by pH-adjusting substances such as: HCl, CH₃COOH, CH₃COO- physiologically adjusted. Further addition of preservatives may be indicated when the primary packaging has proven not to protect against penetration of microorganisms (possibly in the case of non-sterile products). Preservatives such as for example potassium sorbate are employed.

Antimicrobial resistance is becoming a serious problem for the healthcare sector worldwide. For decades, insufficient work has been performed on research of fundamentally new antibiotics, consequently only a few means came to the market. Since then, strain has increased enormously to implement new effective concepts to reduce infections caused by difficult pathogens. Politicians have recognized this need, and extensive funding programs have been launched both nationally and internationally. A mainstay of many publicly funded measures is to search and develop therapeutic agents the effects of which are based on new mechanisms and/or minimization of resistance development.

In medical settings, foreign object infection is associated with increased complication and mortality rates; in this regard, current and prospective antibiotic therapy has reached its limits.

The urgent need for alternative antibiotics is the object of the present invention.

Problems arise due to the low stability of phages in the body, as they are eliminated by phagocytes as foreign objects in a rather short time.

The object of the present invention is to provide technical bacteriophage delivery methods and bacteriophage means, also referred to as bacteriophage depots, which can be applied by technical devices, as well as appropriate application devices for this purpose, to apply appropriate bacteriophage means in a spatiotemporal dosable manner.

Specifically, the object is to apply bacteriophages as an infection prophylaxis in infection therapy to foreign objects and homogenic as well as xenogenic tissues in a different aggregate state, and galenic composition which is biologically active.

Another dependent object is to enable improvement of the bacteriophages to be introduced. This secondary object will be solved by combined application of bacteriophages and endotoxins.

This object or objects will be solved by the bacteriophage means/depots according to the invention as well as bacteriophage means application device and a bacteriophage means manufacturing method according to main claims and independent claims, respectively.

The intracorporal bacteriophage means is formed as a sterile bacteriophage gel, wherein the gel is release-modulatable, or as sterile bacteriophage soft capsules including a gel or as a sterile soft capsule chain with bacteriophage soft capsules including a gel on a monofilamentous hydrolytically degradable thread or on non-degradable material, for example when wicking action is intended, or as a sterile bacteriophage sponge, wherein the sponge is sprayed with a bacteriophage solution or a bacteriophage gel, or a bacteriophage gel is produced by freeze-drying the gel, for example lyophillization.

The naso-pharyngeal and pulmonary bacteriophage means is formed as: a bacteriophage solution or bacteriophage powder, wherein the bacteriophage means mentioned above are nebulizable using at least one of the following variants, namely nebulization through a respirator using a phage nebulization device, nebulization using pressurized gas metered dose inhalers, nebulization using jet nebulizers, nebulization using membrane nebulizers or nebulization using powder inhalers.

The cutaneous bacteriophage means is formed as a sterile bacteriophage powder or a sterile bacteriophage sponge including a bacteriophage gel or a bacteriophage powder as a wound dressing or as a sponge in the form of a freeze-dried bacteriophage gel.

A bacteriophage suture means is a monofilamentous suture formed in a manner circularly sprayed with a bacteriophage solution or with a bacteriophage gel, or it is a polyfilamentous suture wetted with a bacteriophage solution or a bacteriophage gel, the solution or gel being provided on the suture material and/or in the contact zones.

The two-syringe bacteriophage means is characterized in that a first syringe is prepared using a bacteriophage solution and a second syringe is prepared using a gel, wherein the two syringes are connectable to each another especially through a connector, wherein mixing the gel with the bacteriophage solution is possible, and the mixture will be available for application in a syringe. Herein, the gels and also the bacteriophage solutions can be adapted as required. In particular, different gels and/or different bacteriophage solutions may be kept in stock for different combination with each other and mixed accordingly with each other to form an indivitwo bacteriophage solution gel.

The naso-pharyngeal and pulmonary bacteriophage means delivery device, is characterized in that a bacteriophage solution or a bacteriophage powder is nebulizable using at least one of the following devices and the bacteriophage nebulization arrangement is formed as (at least one variant embodiment of one of the following):
a ventilator device including a nebulization device or a pressurized gas-dosage aerosol chamber;
a pressurized gas metered dose inhaler applicator;
a container-inhaler arrangement including a nebulization chamber and a jet nebulizer or a membrane nebulizer;
an inhaler device including a pressurized gas metered dose inhaler;
a powder inhaler.

The bacteriophage sensitive testing application is characterized in that bacteriophages, a bacterial nutrient solution, and a dye, are located in a container, wherein the dye can interact with bacterial cell walls.

In the following, the aforementioned embodiments of the invention will further be described in detail:

A bacteriophage depot or phage means or depot, respectively, is a unit in which at least one bacteriophage or phage is provided as a phage application that maintains stability in a definable period of time following introduction into a body.

In particular, it has been recognized that the bacteriophage is suitable for both treatment and prophylaxis of bacterial diseases or inflammation or fungal diseases, especially because of the very low adverse effects associated therewith. Due to the mechanism of action, which involves multiplication of active bacteriophages only upon the occurrence of infection of the host with an appropriate bacterium or fungus, it is possible to prophylactically administer small amounts of bacteriophages and endotoxins that do not harm the host organism, but which are multiplied in a short time upon the occurrence of bacteria or fungi to be controlled. In general, administration of high doses of bacteriophages is feasible in acute infections because they selectively lyse the pathogenic microorganism without harming the host organism, such as a mammal.

So far, it has been assumed that the immunostimulatory effect of endotoxins causes adverse effects for a patient’s organism, such as increase in temperature (fever) and numerous other pathophysiological effects, wherein release of high doses of endotoxins can even lead to irreversible endotoxin shock. For this reason, endotoxins have so far been undesirable in pharmaceutical compositions, such as those used in bacteria combat. Since it has been assumed that endotoxins are rather harmful and at least irrelevant for any curative success, only endotoxin-free means were considered to be suitable for application so far. In contrast, it has now been surprisingly found that endotoxins in combination with bacteriophages do have a positive effect on the curative success.

The combined application of bacteriophages and endotoxins results in synergistic effect, especially in wound healing. This is attributed to the fact that many inflammatory diseases, such as diabetic foot, are based on three processes, namely a change in the vessels, a change in nerve conduction, and infection. Application of bacteriophages can combat the infection, thereby initiating the overall healing process. The additional presence of endotoxins has a positive effect on the overall healing process. This positive effect is based on the immunostimulatory properties of endotoxins.

In the following examples of technical realizations of bacteriophage applications will be provided. The listing is based on the route of application. The aim of all bacteriophage applications presented is the introduction of therapeutically active bacteriophages for prevention and / or therapy and / or minimization of bacterial infection, wherein, herein, is not the therapeutic procedure that will be focused on but rather the products / applications required therefor. In addition, technologies are provided that relate bacteriophage production, stabilization, or testing the sensitivity of bacteria to existing bacteriophages.

### Variant A - Naso-pharyngeal and Pulmonary Bacteriophage Application - Bacteriophage Solution for Nebulization

Pulmonary bacteriophage application can be performed via several routes. The three main routes are:
1. Nebulization of bacteriophage solution(s) via the ventilator using a phage nebulization device.
2. Nebulization of bacteriophage solution(s) via
   a. pressurized gas metered dose inhalors
   b. nebulization
      b1. jet nebulizer
      b2. membrane nebulizer.
3. Pulmonary application via powder inhalers.
   Option 1:
      Bacteriophage nebulization by ventilator is perfomed using a phage nebulization device, which is to be fitted as an intermediate member to the ventilation hose and is to be connected downstream of the ventilation filter. A flutter valve allows the BPH to be entrained while the patient is being ventilated. Due to the set ventilation pressure, the same amount of bacteriophage solution is always applied per ventilation. The flutter valve closes by reversing the pressure during exhalation, so that no bacteriophage solution is delivered during exhalation. A mesh is located between the BPH solution and the flutter valve which mesh defines the nebulization droplet size. Different meshes can be provided for different therapeutic targets and BPH target regions, thus generating different droplet sizes.
      Since use of the phage nebulization device does not interfere with the airflow, CO₂ measurements as well as nebulization of other therapeutic agents is possible without limitations.
   Option 2:
      The bacteriophage solution is retained in a container which is suitable for a pressurized gas aerosol. The mode of operation is similar to the generally known pressurized gas aerosols.
      In the other nebulizer embodiments, the bacteriophage solution is packaged as a stable solution in single doses and is inserted into the appropriate device before inhalation.
      The therapeutic dose of bacteriophage can be inhaled through all the above-mentioned modes of application - in a manner sealed from the environment. The solution is nebulized into droplet sizes < 5 micrometers thus reaching the bronchial trees up to the alveoli by appropriate inhalation.
   Option 3:
      The bacteriophages are applied to a carrier material, for example lactose, and will be available in bulk or as a single dose (capsule, bilster, ...) for appropriate inhalation. The inhalation devices are operated similar to the powder inhalers which are available on the market, as follows:
      A device mechanism will be used to pierce the capsule, blister or the like, thus releasing the powder. By vigorous inhalation by the patient, the powder is inhaled after having been swirled in the device by obstacles so as to follow the airflow.

### Spray Drying

A suitable carrier material, for example lactose, is dissolved in the bacteriophage solution. The solution is re-converted into the solid aggregate state by spray drying. This results in a solid, amorphous state of the carrier material. This state causes the material to be immediately dissolved in the extracellular fluid, accompanied by release of the bacteriophages.

### Spraying the Bacteriophages onto the Appropriate Carrier Material

Another second option of applying the appropriate bacteriophage solution to the respective carrier material is spraying, wherein the carrier material is moved and transported under nozzles through which the bacteriophage solution will be sprayed. Importantly, the carrier material flows to obtain uniform wetting from all sides.

### Variant B - Intracorporal Application

### 1) Sterile Bacteriophage Gel (Release-Modulated)

The sterile bacteriophage gel can intracorporally be applied in all places. It is prepared from the appropriate bacteriophage solution:

A gel is prepared and sterilized by a gel builder (e.g.: HPMC, ...) without any water portion addition, which will subsequently be added as the bacteriophage solution. Adhesive substances can be added to improve adhesion to different materials (PTFE, ceramic, Dacron, titanium, ...). The bacteriophage solution is sterile-filtered under sterile conditions and maintained in a sterile state in a syringe. The sterilized gel is also kept in a syringe under sterile conditions. For better storage, the two components are mixed by a two-syringe technique by the surgical team shortly before application.

The properties of the resulting gel and thus the bacteriophage release rate therefrom are defined by the amount of gel builder used. For this reason, different gel bases should be retained in syringes. All bacteriophage solutions, which differ in their bacteriophage composition, can be combined with all gel bases using the two-syringe technique.

A surgeon can therefore decide during surgery which viscosity / release and which bacteriophage composition is to be applied. Release in low viscosity gels is fast and release in high viscosity bacteriophage gels is over a longer period of time. The indivitwo syringes (for the gel base and the bacteriophage solution) are each sterile-packaged and are handed over in a sterile state to the non-sterile surgical staff on request. The sterile OR staff mixes the components using the two-syringe technique over a predetermined period of time. The bacteriophage gel is now ready for application.

Other manufacturing options:
using mechanical production, bacteriophage solutions are mixed with an initial portion of the gel and are sterile-filtered. Modulation of this base gel is achieved by appropriate admixing the gel builder under sterile conditions in a downstream production step;
mechanical, sterile filling of the bacteriophage solution and the base gel. The base gel is then sterilized in the final container. Combination is done flexibly and similar to the above-mentioned initial manual procedure.

Other options of sterile bacteriophage gels:
A. The bacteriophages are distributed in a hydrogel matrix in a homogeneously embedded manner. Release from the hydrogel will be determined by the proportion of hydrogel builder in relation to the proportion of water. The product does not interact with skin or mucosal cells, is inert to the human organism and may be used intra-, as well as extra-corporally; possible gel builders include carbomers, cellulose ethers, gelatin, alginates, betonide, highly dispersed silica.
B. Phages are embedded in a lipophilic gel matrix. Release from the hydrogel will be determined by the proportion of lipophilic gel builder relative to the proportion of water. In addition to the antibacterial mechanism, the product is highly moisturizing, supporting the natural wound healing process as in addition to antibacterial therapy/prevention. It can be applied especially extra-corporally, specifically cutaneously; Possible lipophilic gelling agents are highly dispersed silica, aluminum soaps, zinc soaps, especially each having an appropriate lipophilic base, such as mineral oil or liquid triglycerides.
C. The bacteriophages are embedded in an amphiphilic gel matrix. Release from the hydrogel will be determined by the proportion of amphiphilic gelling agent relative to the proportion of water. The product can be applied extra-corporally (see “B.”). Especially, it is for further processing.

All gels can also be introduced in a minimally invasive manner via a syringe. Thus, percutaneous application, e.g.: into an abscess, is also possible.

### 2) Sterile Bacteriophage Soft Capsules

First, a gel is prepared as described above. Herein, the difference is that the final product, i.e. the “final gel” is prepared. Then, this gel, which can vary freely in viscosity, BPH content and BPH composition, is sterile-filled into soft capsules using the rotary die process, the dropping process or other suitable technical processes for generating soft capsules. For example, gelatin is taken as the capsule material. The capsule material is selected according to requirements. Applicable requirements can be the release rate and the scope of therapy. What is meant herein is that lower viscosity forms liquefy more quickly and more strongly in the presence of body fluid and body temperature, thus effluent of these forms from the site of application is stronger and is across a larger area. If the BPH formulation is more likely to be retained at the site of application, a higher viscosity form should be used, which is less rapidly liquefied by body temperature and body fluid, thus remaining at the site of application for a longer period of time. Furthermore, the thickness of the capsule shell and the size of the capsule itself can be varied by the process.

### 3) Sterile Soft Capsule Chain

As described above, soft capsules are manufactured as required. After appropriate manufacture, the capsules are mounted in a fixed distance therebetween on a monofilamentous, hydrolytically degradable thread. This allows application between surgeries, even in body cavities. The capsules are mounted on a non-degradable material for application with, for example, a wicking effect.

### 4) Sterile Bacteriophage Sponge

The gels already presented serve as a basis. They will be freeze-dried (= lyophilized) under exact adjustment and control of the influencing parameters, as well as under sterile conditions. Furthermore, any drying options for the manufacture of a solid bacteriophage application from the appropriate gels are possible.
a. After freeze-drying, the phages are embedded in a solid matrix. Release occurs in a radial manner, i.e. from the outside to the inside, corresponding to a sustained-release mechanism. Release will be determined by the proportion of gel builder in relation to the proportion of water.
b. The bacteriophages are embedded in a now solid lipophilic gel matrix. Release is radial, i.e. from the outside to the inside, corresponding to a sustained-release mechanism. It will be determined by the proportion of gel builder in relation to the proportion of water.
c. The bacteriophages are embedded in a now solid amphiphilic gel matrix. Release is radial, i.e. from the outside to the inside, corresponding to a sustained-release mechanism. It will be determined by the proportion of gel builder in relation to the proportion of water.

This galenic forms result in sustained release of the antibiotic agent. The release rate will be determined by the surface area to volume ratio. Furthermore, collagen fibers can be added to the starting gel to support the appropriate gel matrix as a stabilizer.

### Variant C - Cutaneous Bacteriophage Application / Wound Dressing

### Sterile Bacteriophage "Powder

A bacteriophage product such as the bacteriophage powder for inhalation is sterilely produced and kept in a tightly sealed outer packaging protected from moisture. This product is particularly suitable for weeping wounds such as bums.

### Sterile Bacteriophage Sponge as Wound Dressing

Appropriate to the previously explained bacteriophage sponge. This is covered with an adhesive matrix / adhesive layer the dimensions of which exceed those of the sponge. These adhesive edges, which consist for example of poly-acrylamide as an adhesive component, serve as an adaptation mechanism on the healthy skin.

### Variant D - Bacteriophage Suture Material

In suture manufacture, for example, monofilamentous suture material can be reheated following manufacture to a maximum temperature of 35° C. so that the suture material loosens and, using further application of the heat, the suture is circularly sprayed with a bacteriophage solution or with a bacteriophage gel and the bacteriophage solution is then absorbed during the cooling process.

In the case of polyfilamentous suture material, i.e. suture material consisting of more than one filament, no heating is applied, but only spraying with bacteriophage solution or with a bacteriophage gel is applied, since the solution or gel settles into the contact zones of the indivitwo filaments, remaining on the suture material under subsequent cooling action.

Regardless of manufacture, any suture material can also be placed and packaged in the appropriate gel, with the gel thus surrounding the filament throughout storage.

Especially in the case of hydrolytically cleavable suture material, special care must be taken to ensure that the gel comprises a lipophilic gel builder and that the phages for gel generation are present in a W/O emulsion. The bacteriophage solution provides the water trapped by the oil. This W/O formation may be performed with the help of micelles or emulsifiers.

In the following, examples of technical realizations of bacteriophage applications will be provided. They are based on the route of application. The aim of all bacteriophage applications presented is to introduce therapeutically active bacteriophages for prevention and/or minimization of bacterial infection. In addition, technologies will be provided that facilitate bacteriophage recovery, stabilization or testing sensitivity of bacteria to existing bacteriophages.

Example embodiments of the invention will be described in detail while making reference to the accompanying drawings in the description of figures, and additional embodiments will be shown below which are not shown in the figures. These embodiments are to explain the invention and are not intended to be limiting.

In the figures:

FIG. 1 is a schematic representation of an embodiment of a first bacteriophage application device;

FIG. 2 is a schematic representation of an embodiment of a second pulmonary bacteriophage application device;

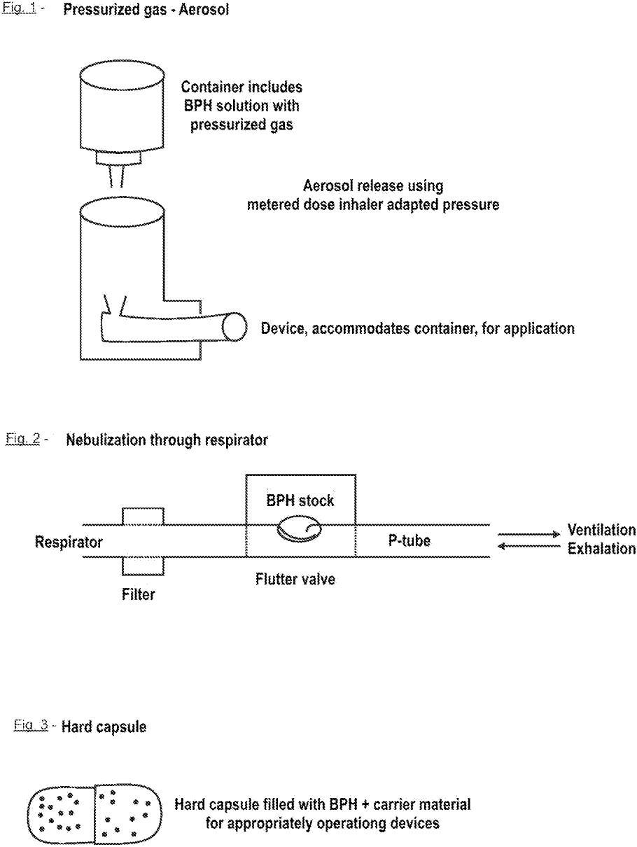

FIG. 3 shows a hard capsule appropriately filled with bacteriophage and carrier material and designed for suitably operating devices;

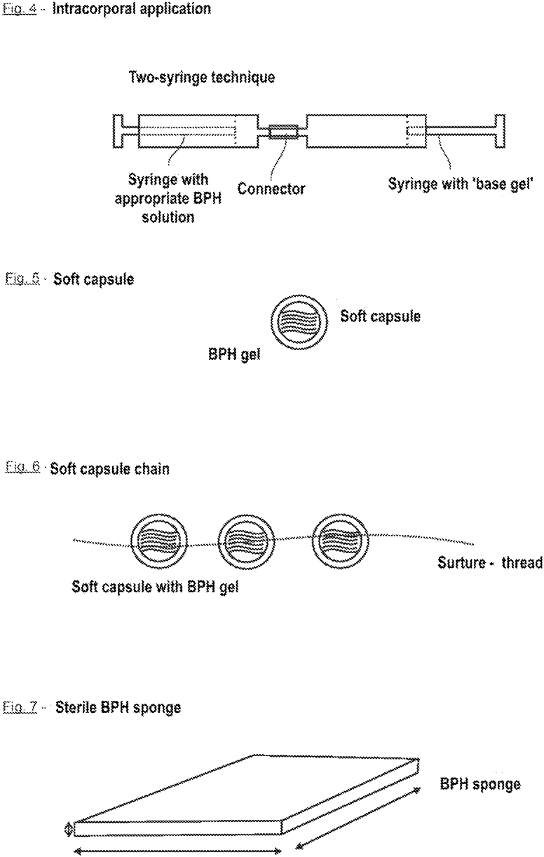

FIG. 4 is another application variant, wherein an intracorporal bacteriophage application is shown herein; FIG. 5 shows another application, wherein a sterile bacteriophage soft capsule application is represented;

FIG. 6 shows a bacteriophage soft capsule chain application;

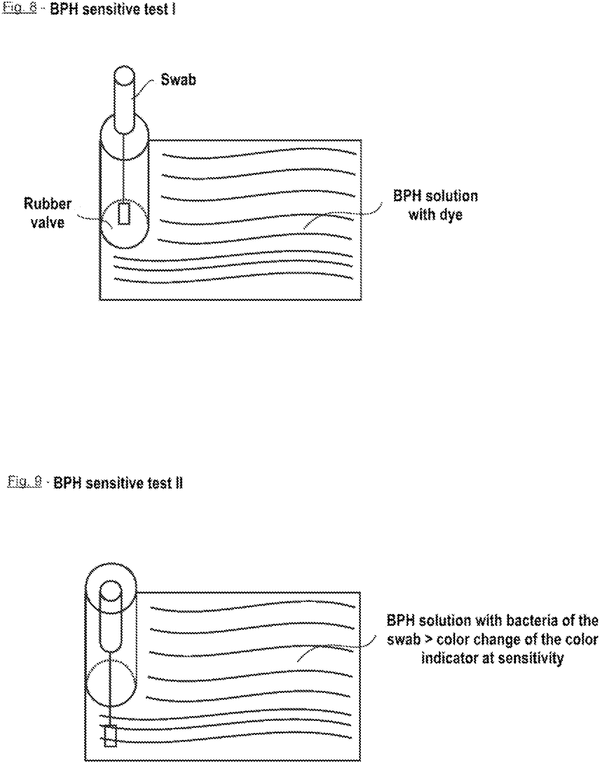

FIG. 7 shows a bacteriophage sponge-gel application variant;

FIG. 8 is a schematic representation of an embodiment of a bacteriophage sensitive testing application shown in the initial state; and

FIG. 9 is a schematic representation of an example embodiment of the bacteriophage sensitive testing application of FIG. 9 in the testing phase.

FIG. 1 is a schematic representation of an example embodiment of a first application, wherein a pulmonary bacteriophage application to be used for the lungs and respiratory tract is shown, wherein a bacteriophage solution for nebulization is provided through pulmonary bacteriophage application devices.

In this regard, the pulmonary bacteriophage application is performed using several types of pulmonary bacteriophage application devices that are directly held against a user’s airway or are connectable to an existing respiratory supply device.

A first pulmonary bacteriophage application device comprises a pressurized gas metering aerosol chamber through which bacteriophage solution(s) are coupleable to the respiratory device. The bacteriophage solution is held in a container compatible with a pressurized gas aerosol.

FIG. 2 shows a second pulmonary bacteriophage application device comprising a nebulization chamber through which bacteriophage solution(s) can be coupled to the respiratory device, the nebulization chamber having j et nebulizers and/or membrane nebulizers.

Bacteriophage nebulization via the respirator is performed using a phage nebulization device, which is to be fitted as an intermediate member in the respiratory tube and is to be connected downstream of the respiratory filter. The bacteriophages are entrained by a flutter valve during ventilation of the patient. Due to the set ventilation pressure, the same amount of BPH solution is constantly applied per ventilation. The flutter valve closes by reversing the pressure during exhalation, so that no bacteriophage solution is delivered during exhalation. Between the BPH solution and the flutter valve is a mesh that sets the nebulization droplet size. Different meshes can be provided for different therapeutic targets and bacteriophage target regions, and thus different droplet sizes can be generated. Since the phage nebulization device does not interfere with the airflow, CO₂ measurements and nebulization of other therapeutic agents remain unrestricted. The bacteriophage solution will preferably be packaged as a stable solution in single doses and will be added to the appropriate device before inhalation.

Another pulmonary bacteriophage application device (not shown in the figures) has a powder inhaler. The bacteriophages are applied to a carrier material and maintained in bulk or as a single dose (capsule, blister, or the like) for appropriate inhalation. The inhalative powder inhaler uses spray drying, thereby dissolving a suitable carrier material, for example lactose, in the bacteriophage solution. The solution is reconverted into the solid aggregate state by spray drying resulting in a solid, amorphous carrier material state. This state causes immediate dissolving of the material by extracellular fluid and accompanying release of the bacteriophages.

Another way of applying the appropriate bacteriophage solution to the respective carrier material is by the spray-on method, wherein the carrier material is brought under nozzles through which the bacteriophage solution is sprayed. It is particularly advantageous for the carrier material to flow to cause allover wetting.

All of the pulmonary bacteriophage applications mentioned above may be used to inhale the therapeutic dose of bacteriophage - sealed from the environment. The solution is especially nebulized in droplet sizes smaller than 5 micrometers, reaching the bronchial trees up to the alveoli by appropriately inhaling.

FIG. 3 shows a hard capsule appropriately filled with bacteriophages and carrier material and designed for suitably operating devices.

FIG. 4 shows another application variant, wherein an intracorporal bacteriophage application is shown producing a sterile bacteriophage gel from a bacteriophage solution which can be applied intracorporally at all places using bacteriophage application devices.

During manufacture, a gel is prepared using a gel builder, for example HPMC or the like, without any water portion of the subsequent bacteriophage solution, and will be sterilized. Adhesive substances can be added for improving adhesion to different materials (PTFE, ceramic, Dacron, titanium, zinc oxide...).

The bacteriophage solution is sterile-filtered under sterile conditions and maintained in a sterile state, e.g. in a syringe. The sterilized gel is maintained under sterile conditions e.g. in a syringe. For better storage, the two components will be mixed in time using a two-syringe technique not earlier than immediately before an application.

The properties of the resulting gel as well as the bacteriophage release rate therefrom are set by the amount of gel builder used. For this reason, different gel bases are kept in stock, e.g. in syringes. All bacteriophage solutions, which differ in their bacteriophage composition, can be combined with all gel bases using the two-syringe technique.

Other ways of gel providing are e.g. a stepwise procedure wherein, using automated production, e.g. bacteriophage solutions are mixed with a first portion of the gel and will be sterile-filtered, and modulation of this basic gel by appropriate admixing of gel builder under sterile conditions in a downstream production step will subsequently be performed.

Furthermore, it is possible to perform automated sterile filling of BPH solution and the base gel, and subsequently sterilizing the base gel in the final container. Contacting the BPH and the base gel may then be performed as required.

Moreover, it is also possible to sterile-fill the BPH solution and base gel by machine and then sterilize the base gel in the final container. Contacting the BPH and the base gel may then be performed as required.

Other ways to provide sterile bacteriophage gels include embedding the phages homogeneously distributed in a hydrogel matrix. Release from the hydrogel is ruled by the proportion of hydrogel builder in relation to the proportion of water. The product does not interact with skin or mucosal cells, will be incorporated into the human organism and may be used intra- and extra-corporally, or the phages can be embedded into a lipophilic gel matrix. Release from the hydrogel will be determined by the proportion of lipophilic gel builder in relation to the proportion of water. In addition to the antibacterial mechanism, the product is highly moisturizing, supporting the intrinsic wound healing process in addition to antibacterial therapy/prevention. It may be applied extra-corporally, especially cutaneously, or the phages may be embedded in an amphiphilic gel matrix. In this case, release from the hydrogel will be determined by the proportion of amphiphilic gel builder in relation to the proportion of water. The product may also be used extra-corporally.

All gels may also be introduced in a minimal-invasive manner via a syringe. Percutaneous application, e.g. into an abscess, now has become possible for the first time.

The viscosity, release rate and BPH composition can immediately be adjusted prior to application. Release in lower viscosity gels is fast and in high viscosity BPH gels release is over a longer period of time.

FIG. 5 shows another application, wherein a sterile bacteriophage soft capsule application is shown, wherein initially a gel is produced, which, contrary to the previously described gel, already provides the final product as a gel, i.e. no further production steps are required. This gel, which may be varied in viscosity, bacteriophage content and composition, as required, is sterile-filled into soft capsules using the rotary die process, the dripping process or other technical processes suitable for generating soft capsules. For example, gelatin is used as the capsule material. The capsule material is selected according to requirements. Applicable requirements may be the release rate and the scope of the therapy. What is meant herein is that low viscosity forms liquefy more quickly and more strongly due to body fluid and body temperature, for example, thus effluent of these forms from the site of application is stronger and is across a larger area. If the BPH formulation is more likely to be retained at the site of application, a higher viscosity form should be used, which is less rapidly liquefied by body temperature and body fluid and remains at the site of application for a longer period of time.

Moreover, different thicknesses of the capsule shell and different sizes of the capsule itself can be used in in the process.

The bacteriophage soft capsule application thus provides a bacteriophage depot or phage depot as a unit in which at least one bacteriophage or phage is provided as a phage application that retains stability after introduction into a body in a manner definable in time.

In addition to the previous embodiments, FIG. 6 shows another application, wherein a sterile bacteriophage soft capsule chain application is provided. As in the bacteriophage soft capsule application, soft capsules are prepared according to appropriate requirements. Following appropriate manufacture, the capsules are mounted in a fixed distance from each other, especially on a monofilamentous hydrolytically degradable thread. This allows application during surgery, even in body cavities.

FIG. 7 shows a variant bacteriophage sponge-gel application. Another application shows a sterile bacteriophage sponge-gel application based on the gels already presented. They are freeze-dried / lyophilized under precise adjustment and control of the influencing parameters, as well as under sterile conditions. Furthermore, all drying options for the production of a solid bacteriophage application from the appropriate gels are allowed.

The phages can be provided after freeze-drying in a form embedded in a solid matrix, embedded in a now solid lipophilic gel matrix or embedded in a now solid lipophilic gel matrix as a bacteriophage sponge-gel application. In each case, release will be radial - from the outside to the inside. This corresponds to a sustained release mechanism. Release will be determined by the proportion of gel builder in relation to the proportion of water.

This galenic form leads to sustained release of the antibiotic agent. The release rate will be determined by the surface area to volume ratio. Furthermore, collagen fibers may be added to the starting gel to support the corresponding gel matrix as a stabilizer.

FIG. 8 and FIG. 9 show a bacteriophage sensitive testing application, wherein a container holds appropriate bacteriophages, a bacterial nutrient solution (liquid), and a dye that interacts with bacterial cell walls. When interacting, the dye is visible A. / the dye is colorless B. A sample, e.g. a swab (sampler included in the set) is placed directly into the container as a test sample. The dye interacts with the bacteria and is A. visible / B. not visible. The testing is incubated for 12 h to 24 h at 36° C. After this time, the sensitivity of the bacteria to the BPH present is indicated in that the solution is less intensely stained A. / is stained B.

### Other Applications

This list of bacteriophage applications is not exhaustive. Combinations of the bacteriophage applications can also be provided and materials can be coated with the bacteriophage applications prior to use, thus generating further applications.

A particularly noteworthy example application for a specific phage application is prosthetics.

Even today, prosthesis infections represent one of the most serious complications of reconstructive surgery. Prosthesis infections, especially with the aorta being affected, often have a lethal course; this technical field in particular is not only highly complicated, but also particularly prone to complications due to the large number of plastics prostheses used.

1-8. (canceled)

9. An intracorporal bacteriophage material or composition, comprising:
a) a sterile bacteriophage gel, wherein said gel is release-modulatable; or
b) sterile bacteriophage soft capsules comprising a gel; or
c) sterile soft capsule chain comprising a bacteriophage soft capsules having a gel on a monofilamentous hydrolytically degradable filament or on a non-degradable material; or
d) a sterile bacteriophage sponge, wherein the sponge is sprayed with a bacteriophage solution or a bacteriophage gel, or a bacteriophage gel is freeze-dried.

10. A naso-pharyngeal and pulmonary bacteriophage material or composition, comprising:
a bacteriophage solution or as a bacteriophage powder,
wherein the bacteriophage material or composition is nebulizable using at least one of the following:
nebulization via respirator using a phage nebulization device;
nebulization via pressurized gas metered dose aerosols;
nebulization via jet nebulizer;
nebulization via membrane nebulizer;
nebulization via powder inhaler.

11. A cutaneous bacteriophage material or composition, comprising
a) a sterile bacteriophage powder; or
b) a sterile bacteriophage sponge including a bacteriophage gel or a bacteriophage powder as wound dressing.

12. A bacteriophage suture material or composition, comprising:
characterized in that
a) a monofilament surture material is circularly sprayed with a bacteriophage solution or with a bacteriophage gel or
b) a polyfilament suture material is wetted with a bacteriophage solution or with a bacteriophage gel, wherein the solution or gel is provided on the suture material and/or in the contact zones.

13. A two-syringe bacteriophage material or compoisition, comprising:
a first syringe is prepared with a bacteriophage solution and a second syringe is prepared with a gel, the two syringes being connectable to each other, wherein mixing of the gel with the bacteriophage solution is possible and this mixture is then present in a syringe ready for application.

14. The two-syringe bacteriophage means according to claim 13, whereindifferent gels and/or different bacteriophage solutions can be kept in stock for different combination among each other and accordingly mixed with each other to form a dedicated bacteriophage solution gel.

15. A naso-pharyngeal and pulmonary bacteriophage material or composition, comprising:
a) a bacteriophage solution or a bacteriophage powder is nebulizable using at least one of the following devices, and the bacteriophage nebulization device is formed as:
a ventilator including a nebulization device or a pressurized gas-dosing aerosol chamber;
a pressurized gas metered dose aerosol applicator
a container inhalation arrangement including a nebulization chamber and a jet nebulizer or a membrane nebulizer;
an inhalation device including a pressurized gas metered-dose inhaler;
a powder inhaler.

16. A bacteriophage sensitive testing application comprising bacteriophages, a bacterial nutrient solution, and a dye are arranged in a container, wherein the dye can interact with bacterial cell walls.
